(19) United States
(12) Patent Application Publication
(10) Pub. No.: US 2005/0240123 A1
(43) Pub. Date: Oct. 27, 2005

(54) Ultrasound medical treatment system and method

(76) Inventors: T. Douglas Mast, Cincinnati, OH (US); Waseem Faidi, Clifton Park, OH (US); Inder Raj S. Makin, Loveland, OH (US); Michael H. Slayton, Tempe, AZ (US); Peter G. Barthe, Phoenix, AZ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/824,196

(22) Filed: Apr. 14, 2004

(51) Int. Cl. A61H 1/00; A61B 8/00

(52) U.S. Cl. 601/2; 600/439

(57) ABSTRACT

An embodiment of an ultrasound medical treatment system includes an ultrasound medical-treatment transducer and a controller. The controller powers the transducer to deliver ultrasound to thermally ablate patient tissue in vivo. In a first expression of the embodiment and/or a first method for thermally ablating patient tissue in vivo which optionally can employ the embodiment, the transducer is powered to deliver ultrasound for or beyond an in vivo treatment time which is a function of an experimentally-determined in vitro treatment time for the same ultrasound acoustic power. In a second expression of the embodiment and/or a second method, the transducer is powered to deliver ultrasound at or above an in vivo ultrasound acoustic power which is a function of an experimentally-determined in vitro ultrasound acoustic power for the same treatment time. In a third expression of the embodiment and/or a third method, the transducer is powered to deliver ultrasound at or above an ultrasound acoustic power threshold which is calculated from an equation.

### FIELD OF THE INVENTION

The present invention relates generally to ultrasound, and more particularly to an ultrasound medical treatment system and method.

### BACKGROUND OF THE INVENTION

Known ultrasound medical systems and methods include deploying an end effector having an ultrasound transducer (powered by a controller) outside the body to break up kidney stones inside the body, endoscopically inserting an end effector having an ultrasound transducer in the rectum to medically destroy prostate cancer, laparoscopically inserting an end effector having an ultrasound transducer in the abdominal cavity to medically destroy a cancerous liver tumor, intravenously inserting a catheter end effector having an ultrasound transducer into a vein in the arm and moving the catheter to the heart to medically destroy diseased heart tissue, and interstitially inserting a needle end effector having an ultrasound transducer needle into the tongue to medically destroy tissue to reduce tongue volume to reduce snoring.

A discrepancy in ultrasound thermal ablation results has been observed between in vitro and in vivo exposures. In the in vivo case, more power (such as, for example, a higher constant power or a greater duty cycle for pulsed power) and/or a longer treatment time were needed. This discrepancy could be explained by in vivo related effects, such as blood perfusion and tissue motion, which tend to remove thermal energy from the heated region. However, this contradicts the fact that the tissue initial temperature in the in vivo exposures (37° C.) was more than that in the in vitro exposures (25° C.). The higher in vivo tissue initial temperature would suggest that less energy is required to reach the ablation target temperature in the in vivo case.

Still, scientists and engineers continue to seek improved ultrasound medical treatment systems and methods.

### SUMMARY OF THE INVENTION

An embodiment of an ultrasound medical treatment system includes an ultrasound medical-treatment transducer and a controller. The controller powers the transducer to deliver ultrasound to thermally ablate patient tissue in vivo. In a first expression of the embodiment and/or a first method for thermally ablating patient tissue in vivo which optionally can employ the embodiment, the transducer is powered to deliver ultrasound for or beyond an in vivo treatment time which is a function of an experimentally-determined in vitro treatment time for the same ultrasound acoustic power. In a second expression of the embodiment and/or a second method, the transducer is powered to deliver ultrasound at or above an in vivo ultrasound acoustic power which is a function of an experimentally-determined in vitro ultrasound acoustic power for the same treatment time. In a third expression of the embodiment and/or a third method, the transducer is powered to deliver ultrasound at or above an ultrasound acoustic power threshold which is calculated from an equation.

Several benefits and advantages are obtained from one or more of the examples of the embodiment and/or methods of the invention. Determining an in vivo treatment time from an experimentally-determined in vitro treatment time for the same ultrasound acoustic power, and/or calculating an in vivo ultrasound acoustic power from an experimentally-determined in vitro ultrasound acoustic power for the same treatment time, allows experimental in vitro treatments to be applied to in vivo treatments despite initial temperature differences of in vivo and in vitro tissue and despite the presence of blood perfusion for in vivo tissue which is not present for in vitro tissue. Calculating an ultrasound acoustic power threshold allows the user to determine if a particular ultrasound medical treatment system has the power required to ablate patient tissue.

The present invention has, without limitation, application in conventional extracorporeal, endoscopic, laparoscopic, intra-cardiac, intravenous, interstitial and open surgical instrumentation as well as application in robotic-assisted surgery.

### BRIEF DESCRIPTION OF THE FIGURES

FIG. 1 is a schematic view of an embodiment of an ultrasound medical treatment system of the invention together with a cross section of a portion of a patient illustrated in the form of in vivo patient tissue to be thermally ablated by the system;

FIG. 2 is a graph of an example of in vivo treatment time versus in vitro treatment time for the same ultrasound acoustic power;

FIG. 3 is a graph of an example of the ratio of in vivo and in vitro ultrasound acoustic power versus the same in vivo and in vitro treatment time;

FIG. 4 is a block diagram of a first method of the invention for thermally ablating patient tissue in vivo which optionally can employ a first expression of the embodiment of the ultrasound medical treatment system of FIG. 1;

FIG. 5 is a block diagram of a second method of the invention for thermally ablating patient tissue in vivo which optionally can employ a second expression of the embodiment of the ultrasound medical treatment system of FIG. 1; and

FIG. 6 is a block diagram of a third method of the invention for thermally ablating patient tissue in vivo which optionally can employ a third expression of the embodiment of the ultrasound medical treatment system of FIG. 1.

### DETAILED DESCRIPTION OF THE INVENTION

Before explaining the present invention in detail, it should be noted that the invention is not limited in its application or use to the details of construction and arrangement of parts and/or steps illustrated in the accompanying drawings and description. The illustrative embodiment, examples, and methods of the invention may be implemented or incorporated in other embodiments, examples, methods, variations and modifications, and may be practiced or carried out in various ways. Furthermore, unless otherwise indicated, the terms and expressions employed herein have been chosen for the purpose of describing the illustrative embodiment and methods of the present invention for the convenience of the reader and are not for the purpose of limiting the invention.

It is understood that any one or more of the following-described examples, methods, implementations, applications, variations, modifications, etc. can be combined with any one or more of the other following-described examples, methods, implementations, applications, variations, modifications, etc. For example, and without limitation, the third method which calculates an ultrasound acoustic power threshold to thermally ablate patient tissue in vivo can be combined with the first method for thereafter determining an in vivo treatment time as a function of an in vitro treatment time.

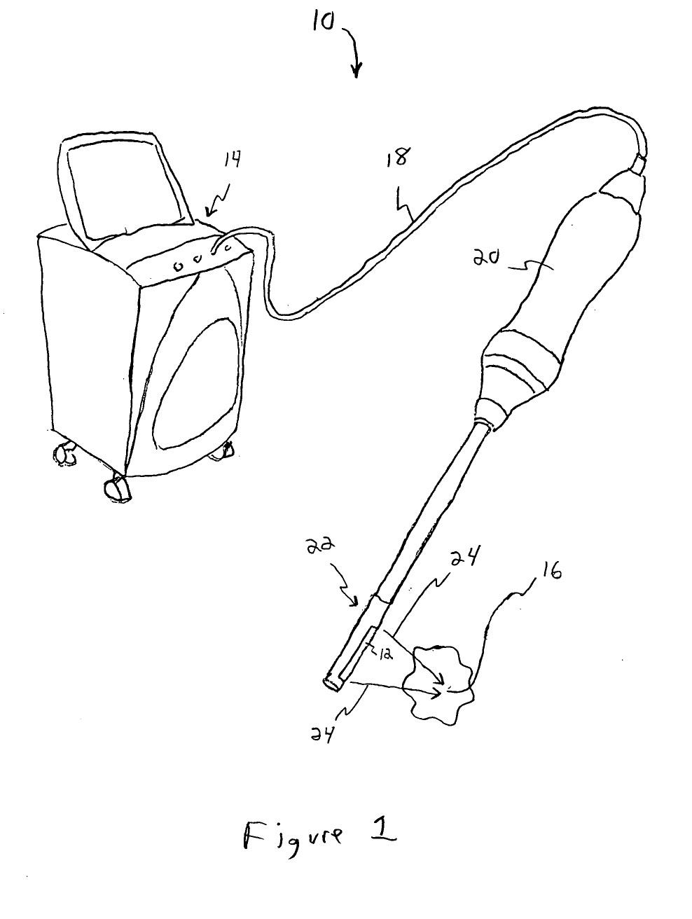

Referring now to the drawings, an embodiment of an ultrasound medical treatment system 10 is shown in FIG. 1. In a first expression of the embodiment of FIG. 1, an ultrasound medical treatment system 10 includes an ultrasound medical treatment transducer 12 and a controller 14. The controller 14 powers the transducer 12 to deliver ultrasound at an ultrasound acoustic power for or beyond an in vivo treatment time to thermally ablate (i.e., create a lesion in) patient tissue 16 in vivo. The controller determines the in vivo treatment time from a function of an experimentally-determined in vitro treatment time for the transducer to deliver ultrasound at the ultrasound acoustic power for the in vitro treatment time to thermally ablate patient tissue in vitro. In one example, the function includes a non-zero blood perfusion rate of the untreated in vivo patient tissue. It is noted that there is no blood perfusion rate for in vitro patient tissue. It additionally is noted that to “deliver ultrasound” is to deliver ultrasound to a site having tissue to be ablated. It is also noted that the in vivo tissue and the in vitro tissue can be from the same patient or different patients (or even from different species of patients having similar tissue).

In one construction of the first expression of the embodiment of FIG. 1, a cable 18 operatively connects the controller 14 to the transducer 12. In one variation, the cable 18 connects the controller 14 to a handpiece 20 which is operatively connected to an end effector 22 which supports the transducer 12. In FIG. 1, the envelope of ultrasound (which is shown as a focused beam but can be an unfocused or divergent beam) from the transducer 12 is indicated by arrowed lines 24.

In one application of the first expression of the embodiment of FIG. 1, the in vivo treatment time is calculated from an equation substantially equivalent to the following Equation #1:

$${time}^{in vivo} = - \frac{ρ}{w} ln [ 1 - \frac{( {T}_{threshold} - {T}_{o}^{in vivo} ) w {time}^{in vitro}}{( {T}_{threshold} - {T}_{o}^{in vitro} ) ρ} ] . \qquad ( 1 )$$

In Equation #1, time^{in vivo} is the in vivo treatment time in seconds to from an in vivo lesion, time^{in vitro} is the in vitro treatment time in seconds to form an in vitro lesion, ρ is the patient tissue density in kilograms per cubic meter, w is the blood perfusion rate in kilograms per cubic meter—seconds, T_{threshold} is the temperature threshold for tissue ablation in degrees Celsius, Tₒ^{in vivo} is the initial in vivo patient tissue temperature in degrees Celsius, and Tₒ^{in vitro} is the initial in vitro patient tissue temperature in degrees Celsius.

In one example of Equation #1, ρ=1060 kg m⁻³, w is 18 kg m⁻³ s⁻¹, T_{threshold} is 60° C., Tₒ^{in vivo} is 37° C., and Tₒ^{in vitro} is 25° C.

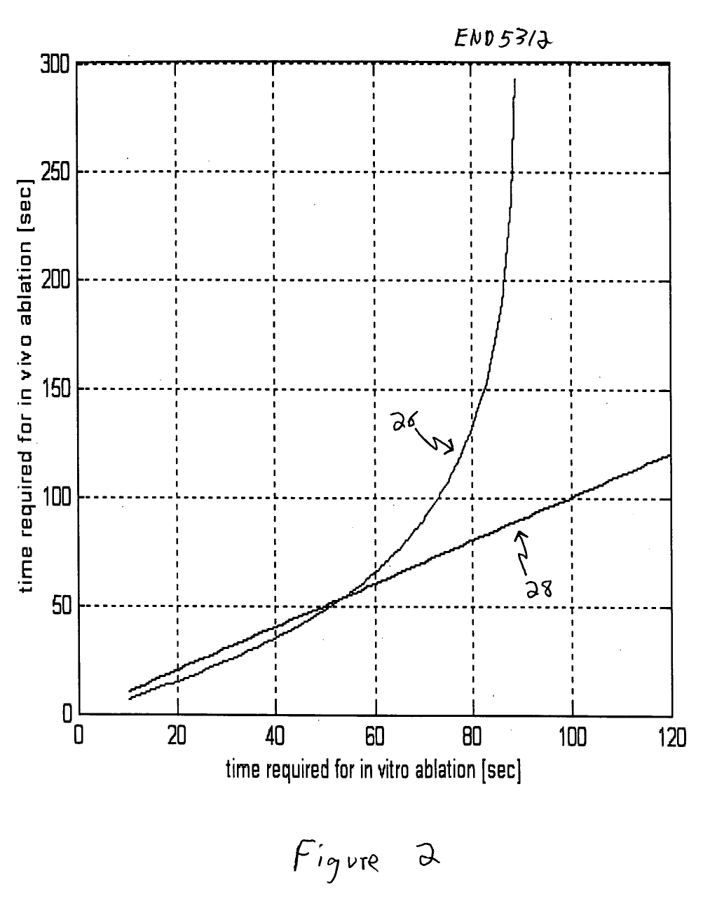

FIG. 2 is a graph of the above-described example of equation #1 plotting in vivo treatment time 26 versus in vitro treatment time for the same in vivo and in vitro ultrasound acoustic power. The straight line 28 of equal in vivo and in vitro treatment times indicates a reference line. For a small in vitro treatment time (i.e., when the heat deposition density is large enough to cause patient tissue ablation in less than 55 seconds), less in vivo treatment time is required. When the heat deposition is small such that 55 seconds or more are required in vitro, blood perfusion becomes dominant and hence in vivo exposures require a longer treatment time than that of their equivalent in vitro exposures. It is noted that for heat deposition density values where approximately 90 seconds or more are required in vitro, no patient tissue ablation can occur in vivo.

In a second expression of the embodiment of FIG. 1, an ultrasound medical treatment system 10 includes an ultrasound medical-treatment transducer 12 and a controller 14. The controller 14 powers the transducer 12 to deliver ultrasound at or above an in vivo ultrasound acoustic power for a treatment time to thermally ablate (i.e., create a lesion in) patient tissue 16 in vivo. The controller determines the in vivo ultrasound acoustic power from a function of an experimentally-determined in vitro ultrasound acoustic power for the transducer to deliver ultrasound at the in vitro ultrasound acoustic power for the treatment time to thermally ablate patient tissue in vitro. In one example, the function includes a non-zero blood perfusion rate of the untreated in vivo patient tissue. It is noted that there is no blood perfusion rate for in vitro patient tissue. It additionally is noted that to “deliver ultrasound” is to deliver ultrasound to a site having tissue to be ablated. It is also noted that the in vivo tissue and the in vitro tissue can be from the same patient or different patients (or even from different species of patients having similar tissue).

In one application of the second expression of the embodiment of FIG. 1, the in vivo ultrasound acoustic power is calculated from an equation substantially equivalent to the following Equation #2:

$${q}^{in vivo} = \frac{( {T}_{threshold} - {T}_{o}^{in vivo} )}{( {T}_{threshold} - {T}_{o}^{in vitro} )} \frac{w time / ρ}{( 1 - {ⅇ}^{- w time / ρ} )} {q}^{in vitro} . \qquad ( 2 )$$

In Equation #2, q^{in vivo} is the in vivo ultrasound acoustic power density (i.e., heat deposition density) in Joules per second—cubic meter to form an in vivo lesion, q^{in vitro} is the in vitro ultrasound acoustic power density in Joules per second—cubic meter to form an in vitro lesion, time is the same in vivo and in vitro treatment time in seconds to form a lesion, ρ is the patient tissue density in kilograms per cubic meter, w is the blood perfusion rate in kilograms per cubic meter—seconds, T_{threshold} is the temperature threshold for tissue ablation in degrees Celsius, Tₒ^{in vivo} is the initial in vivo patient tissue temperature in degrees Celsius, and Tₒ^{in vitro} is the initial in vitro patient tissue temperature in degrees Celsius.

In one example of Equation #2, ρ=1060 kg m⁻³, w is 18 kg m⁻³ s⁻¹, T_{threshold} is 60° C., Tₒ^{vivo} is 37° C., and Tₒ^{in vitro} is 25° C.

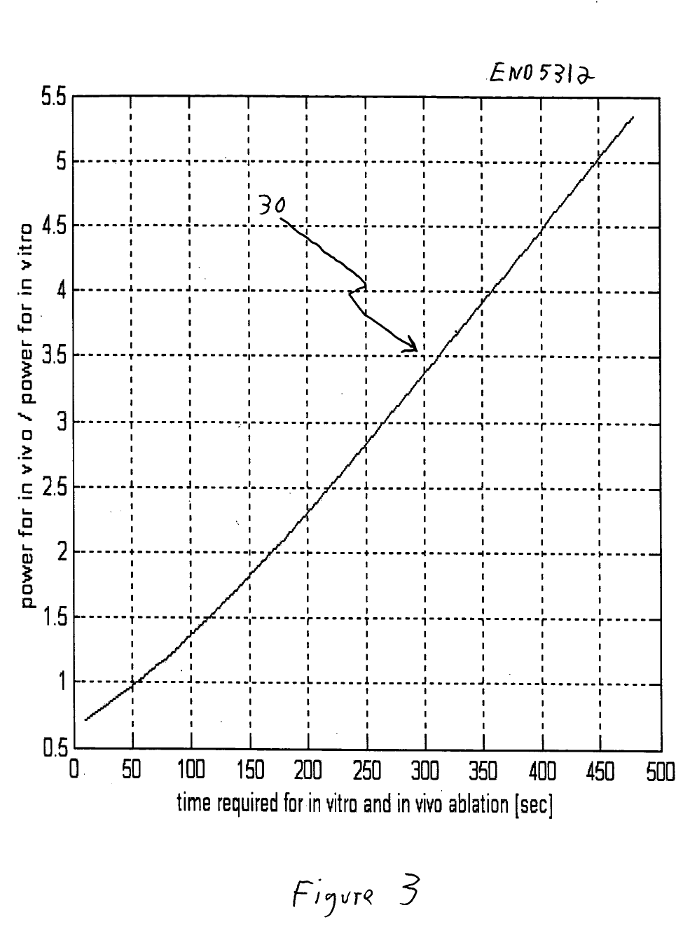

FIG. 3 is a graph of the ratio 30 of in vivo and in vitro ultrasound acoustic power versus the same in vivo and in vitro treatment time from the above-described example of equation #2. It is again noted that for a treatment time longer than 55 seconds, more ultrasound acoustic power (i.e., heat deposition density) is needed in vivo than in vitro.

In a third expression of the embodiment of FIG. 1, an ultrasound medical treatment system 10 includes an ultrasound medical-treatment transducer 12 having an ultrasound emitting area and a controller 14 having a duty cycle (which, in one example, includes a duty cycle of unity). The controller powers the transducer to deliver ultrasound at or above an ultrasound acoustic power threshold to thermally ablate patient tissue 16 in vivo. The controller determines the ultrasound acoustic power threshold from an equation substantially equivalent to the following Equation #3:

$${APO}_{threshold} = F \frac{( {T}_{threshold} - {T}_{b} ) {wc}_{b}}{2 α {\overline{I}}_{ave} DC} \text{Area of transducer.} \qquad ( 3 )$$

In Equation #3, APO_{threshold} is the ultrasound acoustic power threshold in Joules per second to ablate patient tissue in vivo, F is a coefficient to compensate for neglected heat conduction losses in the equation and is between and including 1.05 and 1.15, T_{threshold} is the temperature threshold for tissue ablation in degrees Celsius, T_{b} is the blood temperature in the in vivo patient tissue in degrees Celsius, w is the blood perfusion rate in kilograms per cubic meter—seconds, c_{b} is the patient tissue specific heat capacity in Joules per kilogram—degrees Celsius, “Area of transducer” is the ultrasound emitting area of the transducer 12 in square meters, α is the patient tissue frequency-dependent absorption/attenuation coefficient in Nepers per meter at the transducer frequency, \overline{I}ₐᵥₑ is the intensity gain (local intensity divided by transducer intensity) in the region where the gain is equal to or greater than a certain threshold intensity gain value, and DC is the dimensionless duty cycle of the controller 14 (i.e., DC is the ratio of the therapy on time to the total treatment time for a pulsed controller and DC is 1 [unity] for a non-pulsed controller).

In one example of Equation #3, F=1.1, T_{threshold}=60° C., T_{b}=37° C., c_{b}=3600 J kg⁻°C.⁻¹, the Area of transducer is a nominal area expressed in m², w=18 kg m⁻³ s⁻¹, α=5.75 Np m⁻¹ at the transducer frequency, \overline{I}ₐᵥₑ=1.025, and DC is a nominal value (40%-100%). Applicants had 102 in vivo exposures performed based on equation #3 with the experimental results in 96 of the 102 cases yielding agreement of the theoretical predictions with the experimental results. In 5 of the other 6 cases no lesion was formed although the applied ultrasound acoustic power was more than the calculated threshold. In the remaining case, a lesion was formed although the applied ultrasound acoustic power was less than the calculated threshold.

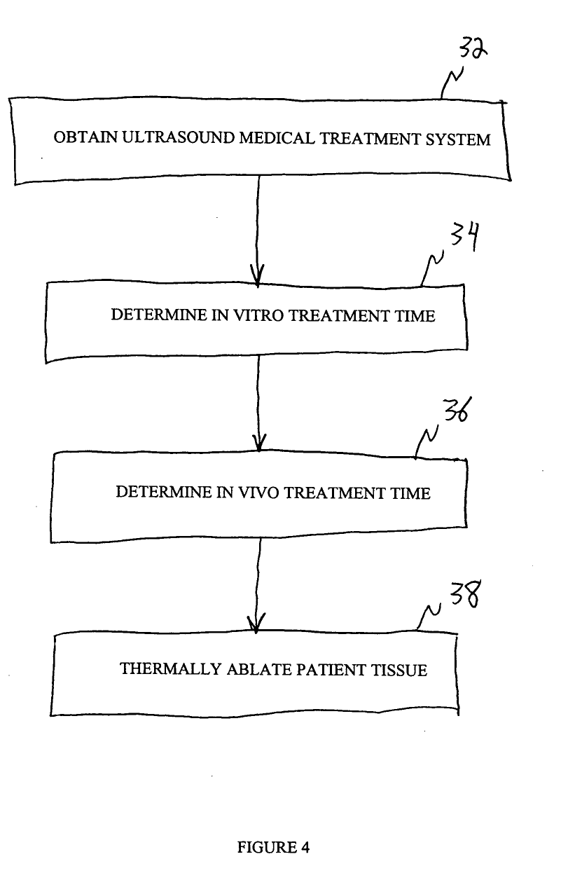

A first method of the invention is shown in block diagram form in FIG. 4 and is for thermally ablating patient tissue 16 in vivo. The first method includes steps a) through d). Step a) is labeled “Obtain Ultrasound Medical Treatment System” in block 32 of FIG. 4. Step a) includes obtaining an ultrasound medical treatment system 10 including an ultrasound medical-treatment transducer 12 and a controller 14 which powers the transducer to deliver ultrasound to thermally ablate patient tissue 16. Step b) is labeled “Determine In Vitro Treatment Time” in block 34 of FIG. 4. Step b) includes experimentally determining an in vitro treatment time for the transducer 12 to be powered by the controller 14 to deliver ultrasound at an ultrasound acoustic power to thermally ablate patient tissue in vitro. Step c) is labeled “Determine In Vivo Treatment Time” in block 36 of FIG. 4. Step c) includes determining (using the controller or otherwise than using the controller) an in vivo treatment time as a function of the in vitro treatment time. Step d) is labeled “Thermally Ablate Patient Tissue” in block 38 of FIG. 4. Step d) includes using the controller 14 to power the transducer 12 to deliver ultrasound at the ultrasound acoustic power for or beyond the in vivo treatment time to thermally ablate patient tissue 16 in vivo.

In one implementation of the first method, step c) is calculated from an equation substantially equivalent to the previously-described Equation #1.

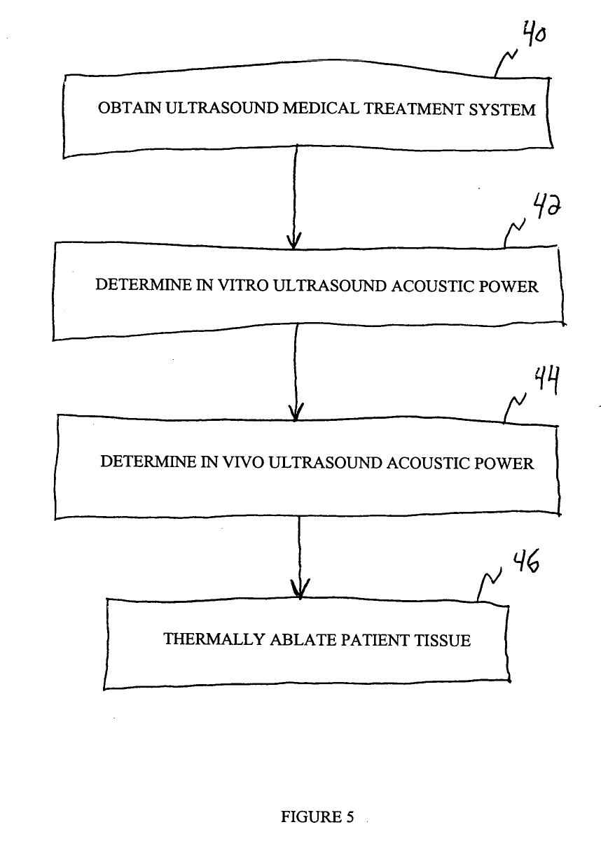

A second method of the invention is shown in block diagram form in FIG. 5 and is for thermally ablating patient tissue 16 in vivo. The second method includes steps a) through d). Step a) is labeled “Obtain Ultrasound Medical Treatment System” in block 40 of FIG. 4. Step a) includes obtaining an ultrasound medical treatment system 10 including an ultrasound medical-treatment transducer 12 and a controller 14 which powers the transducer to deliver ultrasound to thermally ablate patient tissue 16. Step b) is labeled “Determine In Vitro Ultrasound Acoustic Power” in block 42 of FIG. 5. Step b) includes experimentally determining an in vitro ultrasound acoustic power for the transducer 12 to be powered by the controller 14 to deliver ultrasound for a treatment time to thermally ablate patient tissue 16 in vitro. Step c) is labeled “Determine In Vivo Ultrasound Acoustic Power” in block 44 of FIG. 5. Step c) includes determining (using the controller or otherwise than using the controller) an in vivo ultrasound acoustic power as a function of the in vitro ultrasound acoustic power. Step d) is labeled “Thermally Ablate Patient Tissue” in block 46 of FIG. 5. Step d) includes using the controller 14 to power the transducer 12 to deliver ultrasound at or above the in vivo ultrasound acoustic power for the treatment time to thermally ablate patient tissue 16 in vivo.

In one implementation of the second method, step c) is calculated from an equation substantially equivalent to the previously-described Equation #2.

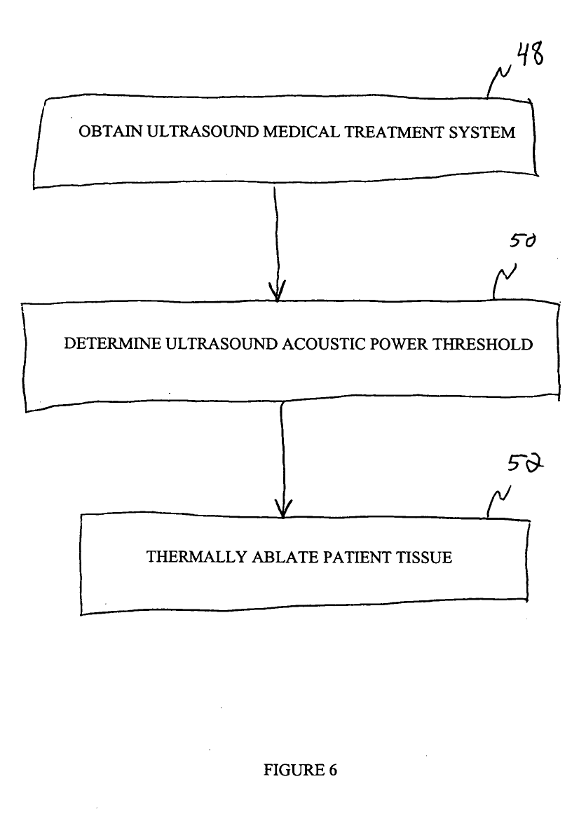

A third method of the invention is shown in block diagram form in FIG. 6 and is for thermally ablating patient tissue 16 in vivo. The third method includes steps a) through c). Step a) is labeled “Obtain Ultrasound Medical Treatment System” in block 48 of FIG. 6. Step a) includes obtaining an ultrasound medical treatment system 10 including an ultrasound medical-treatment transducer 12 having an ultrasound emitting area and a controller 14 having a duty cycle (which, in one example, includes a duty cycle of unity) and powering the transducer to deliver ultrasound to thermally ablate patient tissue 16. Step b) is labeled “Determine Ultrasound Acoustic Power Threshold” in block 50 of FIG. 6. Step b) includes determining an ultrasound acoustic power threshold to thermally ablate patient tissue 16 in vivo, wherein the ultrasound acoustic power threshold is determined from an equation substantially equivalent to the previously-described Equation #3. Step c) is labeled “Thermally Ablate Patient Tissue” in block 52 of FIG. 6. Step c) includes using the controller 14 to power the transducer 12 to deliver ultrasound at or above the ultrasound acoustic power threshold to thermally ablate patient tissue 16 in vivo.

As can be appreciated by those skilled in the art, in one application, the previously-described ultrasound medical treatment system embodiments and methods of the invention are extended to allow treatment plans to be modified not only for the in-vivo versus in-vitro case, but also for cases involving changes in other relevant parameters, such as (without limitation) tissue absorption, initial temperature, and perfusion. The lesioning threshold, required therapy time, and/or required therapy power are all updated based on changes in these parameters using the previously-described or similar formulae. Changes in the parameters, in one illustration, are entered manually, determined from a lookup table based on the tissue type (e.g., liver, kidney, muscle, various tumor types, etc.), or automatically measured by ultrasound or other means.

Also, the methods of the invention, more broadly and collectively expressed as one method, include the step of experimentally determining power and/or timing requirements for one situation (e.g., in vitro) and include the step of determining the corresponding power and/or timing requirements for another situation (e.g., in vivo) using the previously-determined experimental results and a simplified bio-heat model (e.g., considering only bulk tissue heating and perfusion losses and neglecting thermal diffusion as in the cases of the previously-described equations). Likewise, the ultrasound medical treatment system embodiments of the invention, more broadly and collectively expressed as one system embodiment include an ultrasound medical-treatment transducer and a controller. The controller powers the transducer to deliver ultrasound and determines the power and/or timing requirements for a situation using previously-determined experimental results and a simplified bio-heat model.

The ultrasound medical treatment system embodiments and methods of the invention, in one illustration, have the benefit of an a priori estimation of the required source conditions to ensure that a desired tissue effect can be reliably achieved. A technique includes, but is not limited to, programming the controller to have databases/datasets related to the appropriate source conditions for a specific set of tissue effects during treatment. This data is used to modify the output of the controller and implement a certain treatment regime once the user keys-in a particular therapy-related information set. This is achieved, in one example, in an open or a closed feedback loop, by user-modification of source conditions during the treatment cycle, or through operation of the controller in an automated manner.

Several benefits and advantages are obtained from one or more of the examples of the embodiment and/or methods of the invention. Determining an in vivo treatment time from an experimentally-determined in vitro treatment time for the same ultrasound acoustic power, and/or calculating an in vivo ultrasound acoustic power from an experimentally-determined in vitro ultrasound acoustic power for the same treatment time, allows experimental in vitro treatments to be applied to in vivo treatments despite initial temperature differences of in vivo and in vitro tissue and despite the presence of blood perfusion for in vivo tissue which is not present for in vitro tissue. Calculating an ultrasound acoustic power threshold allows the user to determine if a particular ultrasound medical treatment system has the power required to ablate patient tissue.

While the present invention has been illustrated by a description of several methods and several expressions of an embodiment, it is not the intention of the applicants to restrict or limit the spirit and scope of the appended claims to such detail. Numerous other variations, changes, and substitutions will occur to those skilled in the art without departing from the scope of the invention. For instance, the ultrasound methods and system embodiment of the invention have application in robotic assisted surgery taking into account the obvious modifications of such methods, system embodiment and components to be compatible with such a robotic system. It will be understood that the foregoing description is provided by way of example, and that other modifications may occur to those skilled in the art without departing from the scope and spirit of the appended Claims.

1. An ultrasound medical treatment system comprising:
a) an ultrasound medical-treatment transducer; and
b) a controller which powers the transducer to deliver ultrasound at an ultrasound acoustic power for or beyond an in vivo treatment time to thermally ablate patient tissue in vivo, wherein the controller determines the in vivo treatment time from a function of an experimentally-determined in vitro treatment time for the transducer to deliver ultrasound at the ultrasound acoustic power for the in vitro treatment time to thermally ablate patient tissue in vitro.

2. The ultrasound medical treatment system of claim 1, wherein the in vivo treatment time is calculated from an equation substantially equivalent to the following equation:
$${time}^{in vivo} = - \frac{ρ}{w} ln [ 1 - \frac{( {T}_{threshold} - {T}_{o}^{in vivo} ) w {time}^{in vitro}}{( {T}_{threshold} - {T}_{o}^{in vitro} ) ρ} ] ,$$
wherein time^{in vivo} is the in vivo treatment time to from an in vivo lesion, time^{in vitro} is the in vitro treatment time to form an in vitro lesion, ρ is the patient tissue density, w is the blood perfusion rate, T_{threshold} is the temperature threshold for tissue ablation, Tₒ^{in vivo} is the initial in vivo patient tissue temperature, and Tₒ^{in vitro} is the initial in vitro patient tissue temperature.

3. An ultrasound medical treatment system comprising:
a) an ultrasound medical-treatment transducer; and
b) a controller which powers the transducer to deliver ultrasound at or above in vivo ultrasound acoustic power for a treatment time to thermally ablate patient tissue in vivo, wherein the controller determines the in vivo ultrasound acoustic power from a function of an experimentally-determined in vitro ultrasound acoustic power for the transducer to deliver ultrasound at the in vitro ultrasound acoustic power for the treatment time to thermally ablate patient tissue in vitro.

4. The ultrasound medical treatment system of claim 3, wherein the in vivo ultrasound acoustic power is calculated from an equation substantially equivalent to the following equation:
$${q}^{in vivo} = \frac{( {T}_{threshold} - {T}_{o}^{in vivo} )}{( {T}_{threshold} - {T}_{o}^{in vitro} )} \frac{w time / ρ}{( 1 - {ⅇ}^{- w time / ρ} )} {q}^{in vitro} .$$
wherein q^{in vivo} is the in vivo ultrasound acoustic power (i.e., heat deposition density) to form an in vivo lesion, q^{in vitro} is the in vitro ultrasound acoustic power to form an in vitro lesion, time is the same in vivo and in vitro treatment time to form a lesion, ρ is the patient tissue density, w is the blood perfusion rate, T_{threshold} is the temperature threshold for tissue ablation, Tₒ^{in vivo} is the initial in vivo patient tissue temperature, and Tₒ^{in vitro} is the initial in vitro patient tissue temperature Celsius.

5. An ultrasound medical treatment system comprising:
a) an ultrasound medical-treatment transducer having an ultrasound emitting area; and
b) a controller having a duty cycle and powering the transducer to deliver ultrasound at or above an ultrasound acoustic power threshold to thermally ablate patient tissue in vivo, wherein the controller determines the ultrasound acoustic power threshold from an equation substantially equivalent to the following equation:
$${APO}_{threshold} = F \frac{( {T}_{threshold} - {T}_{b} ) {wc}_{b}}{2 α {\overline{I}}_{ave} DC} \text{Area of transducer,}$$
wherein APO_{threshold} is the ultrasound acoustic power threshold to ablate patient tissue in vivo, F is a coefficient to compensate for neglected heat conduction losses in the equation and is between and including 1.05 and 1.15, T_{threshold} is the temperature threshold for tissue ablation, T_{b} is the blood temperature in the in vivo patient tissue, w is the blood perfusion rate, c_{b} is the patient tissue specific heat capacity, “Area of transducer” is the ultrasound emitting area of the transducer, α is the patient tissue frequency-dependent absorption/attenuation coefficient, \overline{I}ₐᵥₑ is the intensity gain in the region where the gain is equal to or greater than a certain value p₁, and DC is the duty cycle of the controller.

6. A method for thermally ablating patient tissue in vivo comprising the steps of:
a) obtaining an ultrasound medical treatment system including an ultrasound medical-treatment transducer and a controller which powers the transducer to deliver ultrasound to thermally ablate patient tissue;
b) experimentally determining an in vitro treatment time for the transducer to be powered by the controller to deliver ultrasound at an ultrasound acoustic power to thermally ablate patient tissue in vitro;
c) determining an in vivo treatment time as a function of the in vitro treatment time; and
d) using the controller to power the transducer to deliver ultrasound at the ultrasound acoustic power for or beyond the in vivo treatment time to thermally ablate patient tissue in vivo.

7. The method of claim 6, wherein the in vivo treatment time in step c) is calculated from an equation substantially equivalent to the following equation:
$${time}^{in vivo} = - \frac{ρ}{w} ln [ 1 - \frac{( {T}_{threshold} - {T}_{o}^{in vivo} ) w {time}^{in vitro}}{( {T}_{threshold} - {T}_{o}^{in vitro} ) ρ} ] ,$$
wherein time^{in vivo} is the in vivo treatment time to from an in vivo lesion, time^{in vitro} is the in vitro treatment time to form an in vitro lesion, ρ is the patient tissue density, w is the blood perfusion rate, T_{threshold} is the temperature threshold for tissue ablation, Tₒ^{in vivo} is the initial in vivo patient tissue temperature, and Tₒ^{in vitro} is the initial in vitro patient tissue temperature.

8. A method for thermally ablating patient tissue in vivo comprising the steps of:
a) obtaining an ultrasound medical treatment system including an ultrasound medical-treatment transducer and a controller which powers the transducer to deliver ultrasound to thermally ablate patient tissue;
b) experimentally determining an in vitro ultrasound acoustic power for the transducer to be powered by the controller to deliver ultrasound for a treatment time to thermally ablate patient tissue in vitro;
c) determining an in vivo ultrasound acoustic power as a function of the in vitro ultrasound acoustic power; and
d) using the controller to power the transducer to deliver ultrasound at or above the in vivo ultrasound acoustic power for the treatment time to thermally ablate patient tissue in vivo.

9. The method of claim 8, wherein the in vivo ultrasound acoustic power in step c) is calculated from an equation substantially equivalent to the following equation:
$${q}^{in vivo} = \frac{( {T}_{threshold} - {T}_{o}^{in vivo} )}{( {T}_{threshold} - {T}_{o}^{in vitro} )} \frac{w time / ρ}{( 1 - {ⅇ}^{- w time / ρ} )} {q}^{in vitro} ,$$
wherein q^{in vivo} is the in vivo ultrasound acoustic power (i.e., heat deposition density) to form an in vivo lesion, q^{in vitro} is the in vitro ultrasound acoustic power to form an in vitro lesion, time is the same in vivo and in vitro treatment time to form a lesion, ρ is the patient tissue density, w is the blood perfusion rate, T_{threshold} is the temperature threshold for tissue ablation, Tₒ^{in vivo} is the initial in vivo patient tissue temperature, and Tₒ^{in vitro} vitro is the initial in vitro patient tissue temperature Celsius.

10. A method for thermally ablating patient tissue in vivo comprising the steps of:
a) obtaining an ultrasound medical treatment system including an ultrasound medical-treatment transducer having an ultrasound emitting area and a controller having a duty cycle and powering the transducer to deliver ultrasound to thermally ablate patient tissue;
b) determining an ultrasound acoustic power threshold to thermally ablate patient tissue in vivo, wherein the ultrasound acoustic power threshold is determined from an equation substantially equivalent to the following equation:
$${APO}_{threshold} = F \frac{( {T}_{threshold} - {T}_{b} ) {wc}_{b}}{2 α {\overline{I}}_{ave} DC} \text{Area of transducer,}$$
wherein APO_{threshold} is the ultrasound acoustic power threshold to ablate patient tissue in vivo, F is a coefficient to compensate for neglected heat conduction losses in the equation and is between and including 1.05 and 1.15, T_{threshold} is the temperature threshold for tissue ablation, T_{b} is the blood temperature in the in vivo patient tissue, w is the blood perfusion rate, c_{b} is the patient tissue specific heat capacity, “Area of transducer” is the ultrasound emitting area of the transducer, α is the patient tissue frequency-dependent absorption/attenuation coefficient, \overline{I}ₐᵥₑ is the intensity gain in the region where the gain is equal to or greater than a certain value p₁, and DC is the duty cycle of the controller; and
c) using the controller to power the transducer to deliver ultrasound at or above the ultrasound acoustic power threshold to thermally ablate patient tissue in vivo.
